(19) United States
(12) Patent Application Publication
(10) Pub. No.: US 2020/0121346 A1
(43) Pub. Date: Apr. 23, 2020

(54) MEDICAL DEVICE

(71) Applicant: Gyrus ACMI, Inc. d/b/a Olympus Surgical Technologies America, Southborough, MA (US)

(72) Inventors: Joel A. Willhite, Memphis, TN (US); David C. Church, Millington, TN (US); Ahmad Alsaffar, Bartlett, TN (US); Jay A. Casey, Memphis, TN (US)

(21) Appl. No.: 16/162,703

(22) Filed: Oct. 17, 2018

(51) Int. Cl. A61B 17/32; A61B 17/22; A61B 17/34

(52) U.S. Cl. CPC A61B 17/32002; A61B 17/320068; A61B 2017/00876; A61B 17/3476; A61B 17/22

(57) ABSTRACT

Provided is a medical device comprising: a solenoid; a permanent magnet having a lumen extending therethrough, the permanent magnet configured to be axially movable within the solenoid; a cannulated shaft configured to run through the lumen of the permanent magnet; an inner tubular member having an open distal end; and an outer tubular member having a distal end and an open window disposed at the distal end, wherein the permanent magnet is configured to reciprocate the cannulated shaft when the solenoid is in electrical communication with a power source, and wherein the open distal end of the inner tubular member and the open window of the outer tubular member are configured to form a cutting tool during an operation of the medical device.

### FIELD

The present disclosure relates generally to a medical device. More particularly, the disclosure relates to a medical device having a permanent magnet designed to drive a working member for use in nasal related surgical operations.

### BACKGROUND

Conventional shavers generally use a rotational motor coupled with a parallel gear train to impart oscillatory motion. For example, PolypVac (manufactured by Laurimed) uses reciprocating cutting motion, presumably powered by suction. Other known ideas are worm gears and ‘levelwind gears’ or ‘diamond screws’. All of these known technologies involve multiple moving components in which a power source, often rotational, translates the input motion to a linear repeating cutting motion. The PolypVac mechanism uses operating room or office-equipped vacuum suction to create and power the cutting motion. Because medical suction devices are of varying quality, strengths and reliabilities, cutting performance and efficiency is often lacking in both power of cutting motion and repeatability of the performance, particularly in the cost-sensitive package of a disposable device. Ostensibly, the mechanical complexity of such a mechanism packaged for one-time use is also subject to quality and reliability problems in the field. Additionally, it's believed that the vacuum pressure used to power the device subtracts from the critical aspiration power needed to engage and pull tissue in to the cutting window. Aspiration can be a critical input feature of a microdebrider with respect to cutting performance.

### SUMMARY

In an embodiment, the present disclosure provides a medical device. In an embodiment, the medical device comprises a solenoid. In an embodiment, the medical device comprises a magnet. In an embodiment, the medical device comprises a permanent magnet. In an embodiment, the medical device comprises a permanent magnet having a lumen extending therethrough. In an embodiment, the medical device comprises a ring or cylindrical or tubular permanent magnet. In an embodiment, the medical device comprises a shaft. In an embodiment, the medical device comprises a cannulated shaft. In an embodiment, the medical device comprises a handpiece. In an embodiment, the medical device comprises an inner tubular member. In an embodiment, the medical device comprises an outer tubular member.

In an embodiment, the present disclosure provides a medical device. In an embodiment, the medical device comprises a solenoid, a permanent magnet and a shaft, wherein the permanent magnet is configured to drive the shaft when the solenoid is in electrical communication with a power source. In an embodiment, the permanent magnet is configured to have a lumen extending through the permanent magnet. In an embodiment, the permanent magnet is configured to be received within the solenoid, and axially movable within the solenoid. In an embodiment, the shaft is configured to be cannulated. In an embodiment, the cannulated shaft is configured to go through the lumen of the permanent magnet. In an embodiment, the lumen of the permanent magnet is configured to receive a portion of the cannulated shaft. In an embodiment, the lumen of the permanent magnet and the solenoid are configured to be co-axial. In an embodiment, the cannulated shaft is configured to be used as a suction line. In an embodiment, the cannulated shaft is configured to drive a working member and to function as a suction line as well.

In an embodiment, the present disclosure provides a medical device. In an embodiment, the medical device comprises a solenoid, a permanent magnet and a cannulated shaft, wherein the cannulated shaft is configured to drive a working member. In an embodiment, the permanent magnet is configured to have a lumen extending through the permanent magnet. In an embodiment, the permanent magnet is configured to be received within the solenoid, and axially movable within the solenoid. In an embodiment, the cannulated shaft is configured to go through the lumen of the permanent magnet. In an embodiment, the lumen of the permanent magnet is configured to receive a portion of the cannulated shaft. In an embodiment, the lumen of the permanent magnet and the solenoid are configured to be co-axial. In an embodiment, the cannulated shaft is configured to be used as a suction line. In an embodiment, the cannulated shaft is configured to drive a working member and to function as a suction line as well.

In an embodiment, the present disclosure provides a medical device. In an embodiment, the medical device comprises a handpiece; a solenoid; a permanent magnet; and a cannulated shaft, wherein the permanent magnet is configured to drive the cannulated shaft when the solenoid is in electrical communication with a power source. In an embodiment, the permanent magnet is configured to have a lumen extending through the permanent magnet. In an embodiment, the permanent magnet is configured to be received within the solenoid, and axially movable within the solenoid. In an embodiment, the cannulated shaft is configured to go through the lumen of the permanent magnet. In an embodiment, the lumen of the permanent magnet is configured to receive a portion of the cannulated shaft. In an embodiment, the lumen of the permanent magnet and the solenoid are configured to be co-axial. In an embodiment, the cannulated shaft is configured to be used as a suction line. In an embodiment, the cannulated shaft is configured to drive a working member and to function as a suction line as well. In an embodiment, the solenoid and the permanent magnet are configured to be disposed within the handpiece.

In an embodiment, the present disclosure provides a medical device. In an embodiment, the medical device comprises a handpiece; a solenoid; a permanent magnet; and a cannulated shaft, wherein the cannulated shaft is configured to drive a working member. In an embodiment, the permanent magnet is configured to have a lumen extending through the permanent magnet. In an embodiment, the permanent magnet is configured to be received within the solenoid, and axially movable within the solenoid. In an embodiment, the cannulated shaft is configured to go through the lumen of the permanent magnet. In an embodiment, the lumen of the permanent magnet is configured to receive a portion of the cannulated shaft. In an embodiment, the lumen of the permanent magnet and the solenoid are configured to be co-axial. In an embodiment, the cannulated shaft is configured to be used as a suction line. In an embodiment, the cannulated shaft is configured to drive a working member and to function as a suction line as well. In an embodiment, the solenoid and the permanent magnet are configured to be disposed within the handpiece.

In an embodiment, the present disclosure provides a medical device. In an embodiment, the medical device comprises a handpiece; an inner tubular member, an outer tubular member; a solenoid; a permanent magnet; and a cannulated shaft, wherein the permanent magnet is configured to drive the cannulated shaft when the solenoid is in electrical communication with a power source. In an embodiment, the permanent magnet is configured to have a lumen extending through the permanent magnet. In an embodiment, the permanent magnet is configured to be received within the solenoid, and axially movable within the solenoid. In an embodiment, the cannulated shaft is configured to go through the lumen of the permanent magnet. In an embodiment, the lumen of the permanent magnet is configured to receive a portion of the cannulated shaft. In an embodiment, the lumen of the permanent magnet and the solenoid are configured to be co-axial. In an embodiment, the cannulated shaft is configured to be used as a suction line. In an embodiment, the cannulated shaft is configured to drive a working member and to function as a suction line as well. In an embodiment, the solenoid and the permanent magnet are configured to be disposed within the handpiece.

In an embodiment, the present disclosure provides a medical device. In an embodiment, the medical device comprises a handpiece; an inner tubular member, an outer tubular member; a solenoid; a permanent magnet; and a cannulated shaft, wherein the cannulated shaft is configured to drive a working member. In an embodiment, the permanent magnet is configured to have a lumen extending through the permanent magnet. In an embodiment, the permanent magnet is configured to be received within the solenoid, and axially movable within the solenoid. In an embodiment, the cannulated shaft is configured to go through the lumen of the permanent magnet. In an embodiment, the lumen of the permanent magnet is configured to partially receive the cannulated shaft. In an embodiment, the lumen of the permanent magnet and the solenoid are configured to be co-axial. In an embodiment, the cannulated shaft is configured to be used as a suction line. In an embodiment, the cannulated shaft is configured to drive a working member and to function as a suction line as well. In an embodiment, the solenoid and the permanent magnet are configured to be disposed within the handpiece. In an embodiment, both the inner tubular member and the outer tubular member are configured to be detachable from the handpiece. In an embodiment, both the inner tubular member and the outer tubular member are configured to be fixedly attached to the handpiece.

In an embodiment, the present disclosure provides a medical device comprising: a housing; a solenoid configured to be stationary inside the housing; a permanent magnet configured to have a lumen extending therethrough; a cannulated shaft configured to go through the lumen of the permanent magnet; an inner tubular member having an open distal end, the inner tubular member configured to be operably connected to the cannulated shaft; and an outer tubular member having a distal end and an open window disposed at the distal end, wherein the open distal end of the inner tubular member and the open window of the outer tubular member are configured to form a cutting tool during an operation of the medical device. In an embodiment, the permanent magnet is configured to be received within the solenoid, and axially movable within the solenoid. In an embodiment, the lumen of the permanent magnet is configured to receive a portion of the cannulated shaft. In an embodiment, the lumen of the permanent magnet and the solenoid are configured to be co-axial. In an embodiment, the cannulated shaft is configured to be used as a suction line. In an embodiment, the cannulated shaft is configured to drive a working member and to function as a suction line as well. In an embodiment, the solenoid and the permanent magnet are configured to be disposed within the housing. In an embodiment, both the inner tubular member and the outer tubular member are configured to be detachable from the housing. In an embodiment, both the inner tubular member and the outer tubular member are configured to be fixedly attached to the housing.

### BRIEF DESCRIPTION OF THE DRAWINGS

FIG. 1 is a schematic view of a medical device in accordance with one aspect of the present disclosure;

FIG. 2 is a sectional view of a medical device in accordance with an exemplary embodiment of the present disclosure;

FIG. 3 is a sectional view of a portion of the medical device shown in FIG. 2.

FIGS. 4A, 4B, and 4C are some exemplary perspective views of the distal end of the tubular section of the medical device as described in FIG. 2.

FIG. 5 is a diagram illustrating a magnet axially activated by a solenoid in accordance with one aspect of the present disclosure.

### DETAILED DESCRIPTION

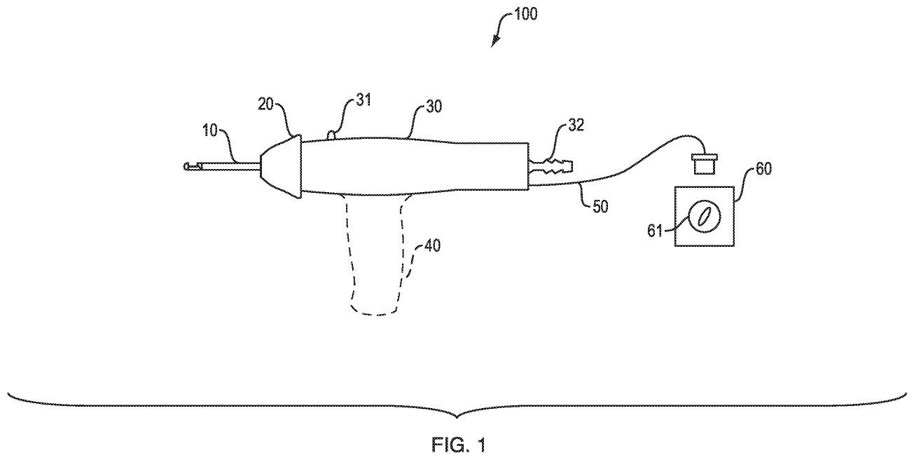

FIG. 1 is a schematic view of a medical device in accordance with one aspect of the present disclosure. The medical device 100 of FIG. 1 includes a tubular section 10, a nosecone 20, a housing 30 (which may also be a handpiece portion by itself), and a possible handpiece 40. The nosecone 20 may be a rotatable nosecone and is between the housing 30 and the tubular section 10. It should be understood that the medical device as described herein may comprise any suitable configurations such as, for example, configurations having a nosecone coupled to an outer member (of the housing), or any other suitable curved or straight debrider configuration which may comprise an irrigation feature. The medical device 100 is configured to be connected to a power supply 60 through a power cable 50. A user-selectable speed dial 61 may also be provided on the power supply 60. The tubular section 10 of the device 100 can be configured with large and/or small tubular members, depending on anatomy and surgeon preference, and can also be adapted for bipolar (preferred) or monopolar radio-frequency (RF) power. An external ESG (electrosurgical generator), for example, may supply the RF power. An activation button 31 may be disposed on the housing 30 in an ergonomic location to power and control the tubular section 10. It should be understood that the activation button may also be provided on a handle portion 40 attached to the housing 30, or activated by a separate footswitch (instead of a button on the housing). Additionally or alternatively, a suction connection 32 may be provided adjacent to the power cable 50.

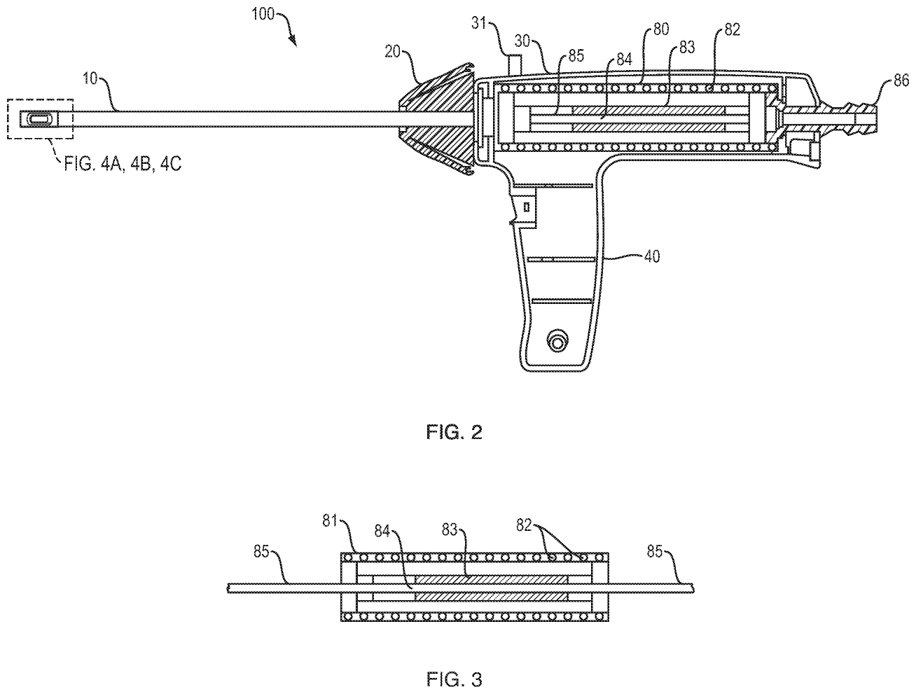

FIG. 2 is a sectional view of a medical device in accordance with one exemplary embodiment of the present disclosure. The medical device 100 of FIG. 2 includes a tubular section 10, a nosecone 20, a housing 30, an activation button 31, a handle 40, a solenoid assembly 80 comprising a casing 81 and a solenoid coil 82 (FIG. 3), a permanent magnet 83, a cannulated shaft 85 with lumen 84, and a suction connector 86.

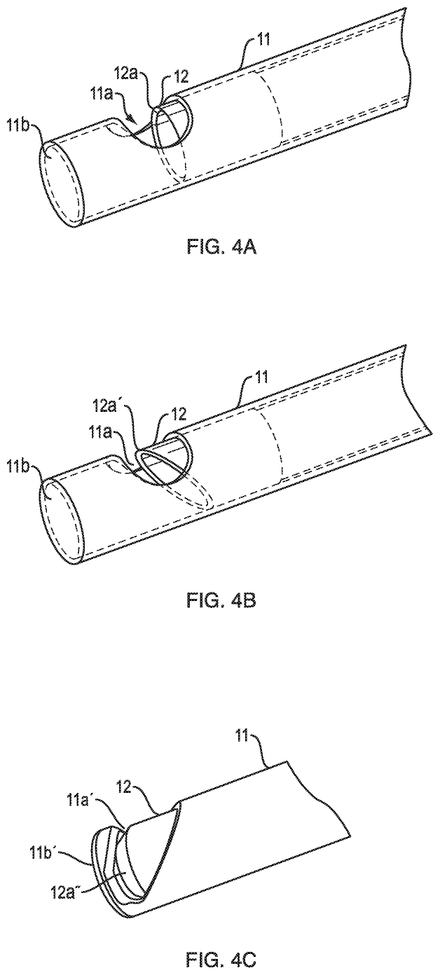

In the above exemplary embodiment, the tubular section 10 may be configured to comprise an outer tubular member 11 and an inner tubular member 12. The outer tubular member 11 is configured to have an open window 11a at its distal end. The outer tubular member 11 may be preferably configured to have a closed end 11b to facilitate better suction of the tubular section 10 (such as shown in FIG. 4A and FIG. 4B). It should be understood that a small hole may be disposed or opened at the closed end 11b (not shown). The small hole may be used to facilitate removal of any cut tissue that might be clogged at or around the open window 11a through fluid irrigation and/or in coordination with suction from a suction source. The small hole may be preferably configured to be capable of toggling between open and closed positions whenever necessary to enhance the suction capacity and/or de-clogging. The inner tubular member 12 may be preferably configured to be open at its distal end. The open distal end of the inner tubular member 12 may be configured to have a circular sharpened edge 12a (such as shown in FIG. 4A) or a beveled sharpened edge 12a′ (such as shown in FIG. 4B) or other suitable sharp edges such as serrated or knife type of edges (not shown). It should be understood that the inner tubular member may also be configured to have a window disposed along the distal end, and the outer tubular member may be configured to have more than one window at its distal end. The open window 11a of the outer tubular member is configured to admit or receive tissue to be cut for a nasal operation. The cut tissue fragments are then drawn through the lumen of the inner tubular member by suction applied at the suction connector 86. The open window 11a may be preferably disposed at a distance of about 1.0 mm to about 1.5 mm from the farthest distal end even though any suitable distance, for example, between 0 and 3 mm, may also be contemplated. It should be understood that any suitable size, dimension, and configuration of the open window 11a may be used. In the above embodiments, the inner diameter of the outer tubular member 11 may be configured to be slightly larger than the outer diameter of the inner tubular member 12 (for example, by approximately 0.002 inches). This allows the inner tubular member 12 to move freely to help minimizing wobbling of the inner tubular member 12 to keep the sharpened edge and the open window 11a closely aligned.

The outer tubular member 11 may be mounted to the housing 30 through the nosecone 20 and acts as a static member, wherein the inner tubular member 12 is received inside the outer tubular member 11, and is configured to be axially movable within the outer tubular member 11. It should be understood that the outer tubular member 11 may be rotatably, detachably or fixedly mounted to the housing 30. The inner tubular member 12 may be configured to be operably connected to the cannulated shaft 85. The open window 11a of the outer tubular member 11 and the sharpened edge such as 12a and 12a′ at the distal end of the inner tubular member 12 provide a cutting feature for the medical device 100, as the magnet 83 is powered by the solenoid assembly to reciprocate the inner tubular member 12 to perform tissue cuts when the coil 82 is electrically or electronically energized (by bringing the sharpened edge such as 12a and 12a′ of the inner tubular member 12 into alignment with the open window 11a of the outer tubular member 11). The above described reciprocating tissue cutting is achieved through a shearing force in a way similar to that exerted by a pair of scissors when the scissors are used to cut an item. It should be understood that both a shearing force and a punching force may be utilized to perform tissue cutting by the medical device as described herein in a manner as illustrated in FIG. 4C. FIG. 4C shows an exemplary design of a tubular section 10 that may utilize both a shearing force and a punching force for cutting tissue. The outer tubular member 11 is configured to have an open window 11b′ very close to the distal end 11b′. In addition to the features of the distal end 11b as described above, the distal end 11b′ may also be configured to have an internal cap type of configuration with a very hard internal surface suitable for accepting a punching force such as stainless steel. This hard internal surface is configured to work with the sharp edged open distal end 12a″ of the inner tubular member 12 to punch off any admitted tissue from the open window 11a′ through the so-called punching or crashing force once the inner tubular member 12 is driven or reciprocating towards the distal end 11b′. The open distal end 12a″ may be configured to be sharp edged, for example, as tapered towards the distal end as shown, or similar to those described above. More particularly, when the medical device is in use, the window 11a′ and the sharp edge 12a″ will generate a shearing force once the inner tubular member is reciprocated by the shaft 85 across the window 11a′ like a pair of scissors, and the shearing force can then be used to cut tissue admitted through the open window 11a′. It should also be understood that because of the close proximity of the open window 11a′ to the distal end 11b′, the sharp edge 12a″ of the inner tubular member 12 will also help pinching off portion of the admitted tissue through its punching force generated through its interaction with the hard internal surface of the close end 11b′ once the inner tubular member 12 is driven towards the distal end 11b′ of the outer tubular member 11. Consequently, FIG. 4C shows a tubular section design that utilizes a shearing force and a punching force to increase its cutting efficiency. It should be understood that the tubular section may also be configured to only utilize the shearing force or only the punching force or both in accordance with the requirements and/or desirability of the medical device. This can be realized through a proper choice of the open window of the outer tubular member such as the location and/or configuration and/or dimension and/or size and/or shape of the open window of the outer tubular member, and optionally or necessarily together with a proper choice of a configuration and dimension of the open distal end of the inner tubular member. It should be understood that a more preferable configuration of the tubular section may be to utilize just the shearing force or a combination of a shearing force and a punching force.

In the above exemplary embodiment, the tubular section 10 may be configured as one piece, and detachable/attachable from/to the housing 30. The nosecone 20 may be configured to rotate the tubular section 10 in addition to being as a coupler between the tubular section 10 and the housing 30. In particular, the nosecone 20 may be configured to be capable of rotating the outer tubular member to a certain desirable position. The activation button 31 is shown in FIG. 2 to be disposed on top of the housing 30. It should be understood that it may also be disposed on the handle 40 or even without an activation switch on the handle. Instead, a separate footswitch may be used for activation.

In the above exemplary embodiment, the solenoid assembly 80 is disposed inside the housing 30. The solenoid assembly 80 comprises a casing 81 and energizing coil 82. The assembly 80 may preferably be fixedly secured inside the housing 30 through known means. The magnet 83 is received within the solenoid assembly 80. The magnet 83 is configured to be axially movable within the solenoid. Preferably, the magnet 83 is configured to be movable completely within the solenoid. The magnet 83 has a hole extending through its entire length. The cannulated shaft 85 has a lumen or opening 84. The cannulated shaft 85 is configured to go through the hole of the magnet 83. The cannulated shaft 85 can be fixedly secured to the magnet 83 as one piece through known means such as high strength glue. The distal end of the cannulated shaft 85 is configured to be operably connected to the inner tubular member 12. The connection may be fixed or detachable. The proximal end of the cannulated shaft 85 is configured to be operably connected to a suction line 86. It should be understood that the magnet 83 and the cannulated shaft 85 may be made into one piece and easily installed into the inside of the solenoid during the manufacturing process. It should also be understood that the solenoid assembly 80, the magnet 83, and the cannulated shaft 85 may be manufactured into one piece as shown in FIG. 3. The whole piece may then be easily installed into the housing 30. It should be further understood that these pieces may be prepared individually, and then assembled into the housing. Once the coil 82 is electrically or electronically energized, it will induce the magnet 83 to move axially, which in turn will activate the shaft 85 to move axially to reciprocate the inner tubular member 12 along the longitudinal axis. Consequently, the open distal end of the inner tubular member will work with the open window of the outer tubular member to admit and then cut tissue. It should be understood that the preferred configuration is to keep the solenoid assembly 80 fixedly secured within the housing 30, and the magnet 83 axially movable in relation to the solenoid assembly 80. It should, however, also be contemplated that the magnet 83 may be configured to be fixed or stationary, and the solenoid assembly 80 is then configured to be axially movable within the housing. It should also be understood that the cannulated shaft may be configured to be outside of the magnet with an offset axis to the longitudinal axis of the solenoid. It may also be contemplated that the magnet may be configured to be solid (no hole or lumen), and to be still coaxial with the solenoid. Under such a situation, the cannulated shaft may then be attached to the side of the magnet.

Further information may also be found in a U.S. application Ser. No. 15/880,998 filed on Jan. 26, 2018, the contents of which are incorporated herein in its entirety by reference.

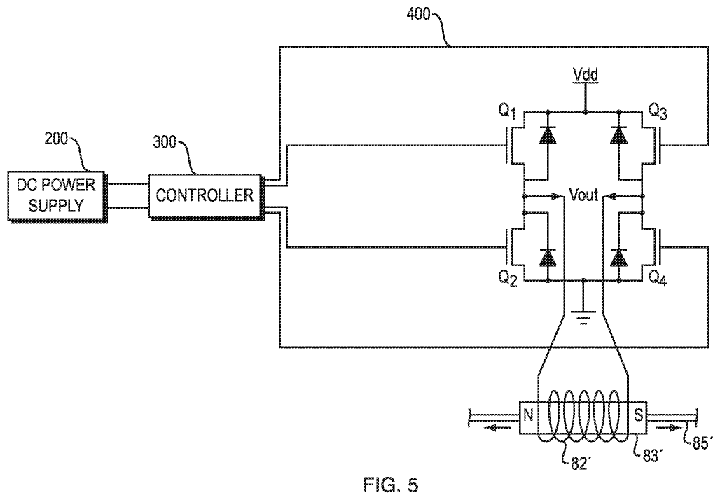

FIG. 5 is a diagram illustrating one example where a magnet is actuated by a solenoid when the solenoid is in electrical or electronic communication with a power source in accordance with one aspect of the present disclosure. More particularly, FIG. 5 illustrates that a controller 300 is configured to control the power output to a solenoid coil 82′ through an H bridge. The controller 300 is connected to a power supply 200 which is configured to energize the solenoid coil 82′ through an H bridge circuit 400 in order to actuate or drive a magnet 83′. The magnet 83′ has a central lumen which receives a cannulated shaft 85′. The controller 300 is configured to be connected to an H bridge 400 which is employed to control the polarity of the output of the DC power supply 200. The H bridge 400 is built with four switches (solid-state or mechanical) with four logic outputs Q1, Q2, Q3, and Q4. It should be understood that only two outputs at a time would be turned on, such as Q1 & Q4, or Q2 & Q3. These outputs will then turn on the appropriate transistors of the H-Bridge. The H-Bridge effectively reverses the polarity of the voltage applied to its output, which is the solenoid. More particularly, When the switches Q1 and Q4 are closed (and Q2 and Q3 are open) a positive voltage will be applied across the motor. By opening Q1 and Q4 switches and closing Q2 and Q3 switches, this voltage is reversed, allowing reverse operation of the magnet 83′. It should be understood that the switches Q1 and Q2 should never be closed at the same time, as this will cause a short circuit on the input voltage source. The same applies to the switches Q3 and Q4. It should be understood that the magnet 83′ may comprise a plurality of smaller magnet rings so long as they are properly aligned even though FIG. 5 shows only one single piece of magnet for clarity. It should also be understood that whether the north pole or the south pole of the magnet is disposed at the distal end or the proximal end should not matter at all since the polarity of the output from the power supply switches every one cycle. The driving or reciprocating force of the coil depends on many factors including the property of the magnet. For example, the number of turns of the coil and the current through them (ampere/turn) primarily determines the force of the solenoid.

By using a permanent magnet as the armature of the solenoid, the direction of movement of the armature is determined by the polarity of the output of the H-Bridge. Only one coil may be required to provide powered movement in both directions even though two or more coils may also be employed when necessary or desirable.

In the above embodiments, the permanent magnet may be neodymium (NdFeB). It may be graded by the “N” number ranging from N35 to N52. The more favorable grade may be N42. It should be understood that a grade of N52 may be used to achieve stronger permanent magnetism. It may be preferable that the magnet is ring magnet. The ring magnet may be made a whole piece or stacked together from smaller pieces. It should be understood that the magnetization direction of the ring magnet is always along the longitudinal direction or the axial direction of the ring magnet. The north pole of the ring magnet may either be proximal or distal with respect to the medical device. The outer diameter of the ring magnet may be about ¼ inch to about 0.4 inch. The outer diameter of the ring magnet may be preferably about 2/8 to about ⅜ inch. The inner diameter may be about ⅛ to about 3/16 inch. The inner diameter of the ring magnet may be preferably about ⅛ inch. The ring magnet may be plated or coated in order to protect the ring magnet from corrosion since most neodymium magnet materials are mostly composed of neodymium, iron and boron. The iron in the magnet will rust if it is not sealed from the environment by some sort of plating or coating. Most of these neodymium magnets may be plated in nickel-copper-nickel. Some of them may also be plated in gold, silver, or black nickel, while others are coated in epoxy, plastic or rubber. The ring magnet may be further protected by a polymer casing or tubing. In the above embodiments, the ring magnet is configured to be movable within the solenoid. In the above embodiments, the magnet may be preferably configured to be axially movable completely within the solenoid.

In the above embodiments, the permanent magnet may be configured to drive or reciprocate the cannulated shaft at a frequency of about 3 to 30 Hz. In the above embodiments, the magnet may be preferably configured to drive or reciprocate the cannulated shaft at a frequency of about 3 to 10 Hz. In the above embodiments, the magnet may be configured to drive or reciprocate the working member at a frequency of about 3 to 30 Hz. In the above embodiments, the magnet may be preferably configured to drive or reciprocate the working member at a frequency of about 3 to 10 Hz. In the above embodiments, the magnet may be configured to drive or reciprocate the inner tubular member at a frequency of about 3 to 30 Hz. In the above embodiments, the magnet may be preferably configured to drive or reciprocate the inner tubular member at a frequency of about 3 to 10 Hz.

In the above embodiments, the solenoid may be sized and/or dimensioned to fit into a handpiece and also to accept a magnet. In the above embodiments, the solenoid may be configured to have an outer diameter of about 0.75 inch to about 1.0 inch. The solenoid may be preferably configured to have an outer diameter of about 1 inch. In the above embodiments, the solenoid may be configured to have an inner diameter that is capable of receiving a ring magnet. In the above embodiments, the solenoid may be configured to have a length of about 2 inch to about 3 inch. In the above embodiments, the solenoid may be configured to have a casing. It should be understood that the solenoid should be configured to be capable of generating a power of about 50 watts to about 150 watts. In the above embodiments, the solenoid may be configured to be capable of generating about 2 pound of force. In the above embodiments, the solenoid may be configured to be capable of generating about 6 pound of force. In the above embodiments, the solenoid may be configured to be stationary.

In the above embodiments, the inner tubular member has a distal portion and a proximal portion. The proximal portion may be configured to be operably connected to the cannulated shaft. The distal portion may be configured to have an open distal end or tip. The open distal end may be configured to be any suitable sharp edge for cutting purpose. For example, it may be configured to be circular with sharp edge. It may be configured to have beveled sharp edge. The bevel angle may be, for example, at 45° degree in relation to the longitudinal axis. The open distal end or tip of the inner tubular member may be made of any suitable metals such as 300 and 17-4 stainless steel. The rest of the inner tubular member may be made of the same material as the tip or of a different material such as a polymeric material. If the tip and the remaining portion of the inner tubular member are made of different materials, the metal tip and the remaining portion of the inner tubular member may be fused together with known means in the art such as glue and press-fit. The inner tubular member is preferably configured to be flexible. In the above embodiments, the outer tubular member has a distal portion and a proximal portion. The distal portion may be configured to have an open window. The proximal portion is configured to be fixedly or detachably connected to the handpiece. The outer tubular member may be made of any suitable materials such as 300 or 17-4 stainless steel, other metallic, and even polymeric materials. The outer tubular member may be preferably made to be malleable. In the above embodiments, the inner tubular member may be configured to be movable relative to the outer tubular member. In the above embodiments, the outer tubular member may be configured to be stationary. In the above embodiments, the open distal end of the inner tubular member and the open window of the outer tubular member are configured to form a cutting tool or device during an operation of the device. It should also be understood that a bit larger open window may help admitting more tissue for cutting while a smaller window may facilitate a better suction. Accordingly, the open window of the outer tubular member may be sized/shaped/configured/dimensioned in accordance with the needs and/or functions of the medical device.

In the above embodiments, the open window of the outer tubular member may be configured to have an outer surface edge and an inner surface edge. The outer surface edge may be preferably configured to be smooth in order for it not to cause damage or harm to the tissue/passage way when the device is in the process of being inserted. The inner surface edge, in particular towards the distal end, may be preferably configured to be sharp so that the sharp edge may form a more efficient cutting tool with the sharp edge of the open distal end of the inner tubular member. Similarly, the open distal end of the inner tubular member may be configured to have an outer surface edge and an inner surface edge. The outer surface edge may be preferably configured to be sharp in order for it to form a more efficient cutting tool with the inner surface edge of the outer tubular member.

In the above embodiments, the tubular section may also be configured to comprise more than two tubular members such as an inner tubular member, an outer tubular member and an intermediary tubular member. In such embodiments, either the inner tubular member or the intermediary tubular member may be configured to be operably connected to the cannulated shaft of the medical device. The inner tubular member or the intermediary tubular member may also be configured to be operably connected to a motor. In such embodiments, the intermediary tubular member may be configured to have an open window at its distal end. In such embodiments, the outer tubular member may be configured to completely cover the open window area of the intermediary tubular member for safe insertion of the medical device.

In the above embodiments, the cannulated shaft and the lumen of the magnet are configured to be concentric or coaxial. In the above embodiments, the cannulated shaft is configured to be sized and dimensioned to fit into the lumen of the magnet and to be strong to withstand the force expected for a medical device, in particular, for a nasal medical device. In the above embodiments, the cannulated shaft may be configured to be fixed to the permanent magnet by high strength adhesive and/or other means known in the art. In an embodiment, the cannulated shaft may be configured to be outside of the magnet. In an embodiment, the cannulated shaft may be configured to be attached to the outside of the magnet if the magnet is configured to have no hole or lumen. In an embodiment, the shaft may be configured to be solid wherein the suction may be configured to be through another line.

In the above embodiments, a DC power supply may provide the power for the solenoid. The power supply may be from 12V to 48V. More particularly, the power supply may be from 12V, 18V, 24V, 30V, 36V, 42V or 48V. In the above embodiments, the reciprocating frequency may be from 3 to 30 Hz. The solenoid may have a total length of about 2 inch to about 3 inch. In the above embodiments, the solenoid may have a resistance of about 6 to about 30 ohms.

In all the above embodiments, it should be understood that the connections, the sealing, and securing between the outer tubular member, the inner tubular member, the permanent magnet, the cannulated shaft, and the solenoid may be achieved through the necessary supporting bearings, connectors, couplers, springs, and other means known in the art. It should also be understood that the connections, the sealing, and securing between other components such as nosecone may be similarly achieved. In the above embodiments, the medical device may be assembled in accordance with the methods known in the art.

In the above embodiments, the medical device may be configured for use in the removal of nasal polyps, sub-mucosal debulking of turbinate, and functional endoscopic sinus surgery (FESS), primarily in the office environment and/or cost-sensitive regions. It may be a disposable debrider or shaver. In the above embodiments, the medical device may provide several benefits such as more precise cutting, lower blood loss or less bleeding in comparison with a medical device with rotary or oscillatory cutting mode since the reciprocating mode only allows the tissue admitted or sucked into the open window of the outer tubular member to be cut while a rotary or oscillatory mode may cause unnecessary or undesired tissue cut because of its pulling impact during an operation.

In another embodiment, the present disclosure also provides a method of cutting tissue. In an embodiment, the method includes providing a medical device having an inner tubular member and an outer tubular member. In an embodiment, the method includes inserting the medical device into a treatment site and positioning the medical device properly. In an embodiment, the method includes turning on the device to cut tissue. More particularly, the method includes cutting tissue by reciprocating the inner tubular member in relation to the outer tubular member wherein the outer tubular member and the inner tubular member work to admit and cut tissue. It should be understood that once the medical device is turned on, the open window of the outer tubular member constantly aligns with the open distal end of the inner tubular member to capture/admit and cut the tissue. It should also be understood that once the device is powered off, the distal end of the inner tubular member and the distal end of the outer tubular member may be configured to form a closed configuration to keep the device safe for the patient.

It is understood that the above description is intended to be illustrative and not restrictive. Many embodiments as well as many applications besides the examples provided will be apparent to those of skill in the art upon reading the above description. The scope of the teachings should, therefore, be determined not with reference to the above description, but should instead be determined with reference to the appended claims, along with the full scope of equivalents to which such claims are entitled. The disclosures of all articles and references, including patent applications and publications, are incorporated by reference for all purposes. The omission in the following claims of any aspect of subject matter that is disclosed herein is not a disclaimer of such subject matter, nor should it be regarded that the inventors did not consider such subject matter to be part of the disclosed inventive subject matter.

The principles of the present disclosure may be better understood with reference to the drawings and the accompanying descriptions, wherein like reference numerals have been used throughout to designate identical or similar elements. It should be understood that these drawings are not necessarily are drawn to scale. They are presented just for illustrative purposes only, and are not intended to limit the scope of the disclosure. Examples of materials, dimensions, and constructions are included for some elements. Those of ordinary skill in the art should understand that many of the examples provided have suitable alternatives and these alternatives should also be considered within the scope of this disclosure. Moreover, certain terminology is used herein for convenience only and is not to be taken as a limitation on the present disclosure.

The explanations and illustrations presented herein are intended to acquaint others skilled in the art with the disclosure, its principles, and its practical applications. Those skilled in the art may adapt and apply the disclosure in numerous forms, as may be best suited to the requirements of a particular use. The specific embodiments of the present disclosure as set forth are not intended to be exhaustive or limiting of the invention. The scope of the invention should be determined not with reference to the above description, but should be determined with reference to the appended claims, along with the full scope of equivalents to which such claims are entitled.

The terms “one embodiment”, “an embodiment”, “another embodiment”, “some embodiments”, “other embodiments”, “above embodiment”, and similar expressions indicate that the embodiment or embodiments described may include a particular feature, structure, or characteristic, but every embodiment may not necessarily include the particular feature, structure, or characteristic. Moreover, such phrases are not necessarily referring to the same embodiment. Furthermore, when a particular feature, structure, or characteristic is described in connection with an embodiment, it would be within the knowledge of one skilled in the art to incorporate such feature, structure, or characteristic into other embodiments, whether or not explicitly described, unless clearly stated to the contrary. That is, the various individual elements described below, even if not explicitly shown in a particular combination, are nevertheless contemplated as being combinable with each other to form other additional embodiments or to complement and/or enrich the described embodiment or embodiments, as would be understood by one of ordinary skill in the art.

The articles “a”, “an” and “the” are used herein to refer to one or to more than one (i.e. to at least one) of the grammatical object of the article unless otherwise clearly indicated by contrast. By way of example, “an element” means one element or more than one element.

The term “including” is used herein to mean, and is used interchangeably with, the phrase “including but not limited to”. The term “or” is used herein to mean, and is used interchangeably with, the term “and/or”, unless context clearly indicates otherwise.

The term “such as” is used herein to mean, and is used interchangeably, with the phrase “such as but not limited to”. Unless specifically stated or obvious from context, as used herein, the term “about” is understood as within a range of normal acceptance in the art, for example within standard deviations of the mean.

The term “proximal” is herein used to mean a position or direction closest to a user of the device and is in a position or direction opposite to the term “distal”.

The term “distal” is herein used to mean a position or direction furthest away from a user of the device and is a position or direction opposite to the term “proximal”.

The term “cannulated” used throughout the specification refers to a general ‘tube’ or ‘tubular’, or ‘hollowed out cylindrical’ shape, or any general cylinder shape having an outside diameter and an inside diameter, for example.

All numeric values are herein assumed to be modified by the term “about” whether or not explicitly indicated. The term “about”, in the context of numeric values, generally refers to a range of numbers that one of skill in the art would consider equivalent to the recited value (i.e., having the same function or result). In many instances, the term “about” may include numbers that are rounded to the nearest significant figure. Other uses of the term “about” (i.e., in a context other than numeric values) may be assumed to have their ordinary and customary definition(s), as understood from and consistent with the context of the specification, unless otherwise specified. Even more specifically, “about” can be understood as within 10%, 9%, 8%, 7%, 6%, 5%, 4%, 3%, 2%, 1%, 0.5%, 0.1%, 0.05%, or 0.01% of the stated value. Unless otherwise stated, all ranges include both endpoints and all numbers between the endpoints. The use of “about” or “approximately” in connection with a range applies to both ends of the range. Thus, “about 10 to 30” is intended to cover “about 10 to about 30”, inclusive of at least the specified endpoints.

1. A medical device comprising:
a solenoid;
a permanent magnet having a lumen extending therethrough, the permanent magnet configured to be received within the solenoid; and
a cannulated shaft extending through the lumen of the permanent magnet, where the cannulated shaft comprises a proximal end extending out of a proximal end of the solenoid and configured to be operably connected to a suction line, and where the cannulated shaft comprises a distal end extending out of a distal end of the solenoid and configured to be connected to an inner tubular member,
wherein the solenoid is configured to drive the magnet when the solenoid is in electrical communication with a power source such that the permanent magnet and the solenoid are configured to move the cannulated shaft in a first direction in the medical device with a first energization of the solenoid, and such that the permanent magnet and the solenoid are configured to move the cannulated shaft in a second opposite direction in the medical device with a second different energization of the solenoid to thereby reciprocatingly move the cannulated shaft in the medical device.

2. The medical device of claim 1, wherein the medical device further comprises the inner tubular member having an open distal end, where the inner tubular member is removably connected to the cannulated shaft.

3. (canceled)

4. The medical device of claim 2, wherein the medical device further comprises an outer tubular member having a distal end and an open window disposed at the distal end, where the outer tubular member is on the inner tubular member.

5. The medical device of claim 4, wherein the open distal end of the inner tubular member and the open window of the outer tubular member are configured to form a cutting tool during an operation of the medical device.

6. The medical device of claim 5, wherein the outer tubular member is configured to be stationary.

7. The medical device of claim 5, wherein the outer tubular member is configured to remain stationary relative to a housing of the medical device.

8. The medical device of claim 1, wherein the permanent magnet is a ring or tubular magnet.

9. The medical device of claim 1, wherein the solenoid is configured to be stationary relative to a housing of the medical device.

10. The medical device of claim 1, wherein the medical device further comprises a housing.

11. A medical device comprising:
a solenoid;
a permanent magnet having a lumen extending therethrough, the permanent magnet configured to be axially movable within the solenoid;
a cannulated shaft extending through the lumen of the permanent magnet, where the cannulated shaft comprises a proximal end extending out of a proximal end of the solenoid and configured to be operably connected to a suction line, and where the cannulated shaft comprises a distal end extending out of a distal end of the solenoid; and
a working member operably connected to the distal end of the cannulated shaft,
wherein the solenoid is configured to drive the magnet when the solenoid is in electrical communication with a power source such that the permanent magnet and the solenoid are configured to move the cannulated shaft in a first direction in the medical device with a first energization of the solenoid, and such that the permanent magnet and the solenoid are configured to move the cannulated shaft in a second opposite direction in the medical device with a second different energization of the solenoid to thereby reciprocate the cannulated shaft in the medical device, wherein the cannulated shaft is configured to drive the working member when the solenoid is in electrical communication with a power source.

12. The medical device of claim 11, wherein the working member comprises an outer tubular member and an inner tubular member.

13. The medical device of claim 12, wherein the inner tubular member has an open distal end and the outer tubular member has an open window.

14. The medical device of claim 13, wherein the open distal end of the inner tubular member and the open window of the outer tubular member are configured to form a cutting tool during an operation of the medical device.

15. The medical device of claim 12, wherein the inner tubular member is configured to be operably connected to the cannulated shaft.

16. A medical device comprising:
a handle;
a solenoid disposed inside the handle;
a permanent magnet having a lumen extending therethrough, the permanent magnet configured to be axially movable within the solenoid; and
a cannulated shaft extending through the lumen of the permanent magnet, where the cannulated shaft comprises a proximal end extending out of a proximal end of the solenoid and configured to be operably connected to a suction line, and where the cannulated shaft comprises a distal end extending out of a distal end of the solenoid;
an inner tubular member having an open distal end, where the inner tubular member is connected to the distal end of the cannulated shaft; and
an outer tubular member on the inner tubular member, where the outer tubular member comprises a distal end and an open window disposed at the distal end,
wherein the solenoid is configured to drive the magnet when the solenoid is in electrical communication with a power source such that the permanent magnet and the solenoid are configured to move the cannulated shaft in a first direction in the medical device with a first energization of the solenoid, and such that the permanent magnet and the solenoid are configured to move the cannulated shaft in a second opposite direction in the medical device with a second different energization of the solenoid to thereby reciprocate the cannulated shaft in the medical device.

17. The medical device of claim 16, wherein the open distal end of the inner tubular member and the open window of the outer tubular member are configured to form a cutting tool during an operation of the medical device.

18. The medical device of claim 16, wherein the inner tubular member is removably connected to the cannulated shaft.

19. The medical device of claim 16, wherein the outer tubular member is configured to remain stationary relative to the handle.

20. (canceled)

21. The medical device of claim 1 where the solenoid extends past opposite ends of the permanent magnet for all locations of the permanent magnet inside the solenoid.

22. The medical device of claim 1 where the solenoid is housed in a casing, and where the casing comprises a suction connector extending out of a proximal end of the casing, where the suction connector is configured to have the suction line directly mounted thereto.

23. The medical device of claim 1 further comprising an H bridge circuit connected to the solenoid.
